(19) United States
(12) Patent Application Publication
(10) Pub. No.: US 2005/0032144 A1
(43) Pub. Date: Feb. 10, 2005

(54) Method for selective conjugation of analytes to enzymes without unwanted enzyme-enzyme cross-linking

(76) Inventors: Vincent C Lombardi, Reno, NV (US); David A. Schooley, Reno, NV (US)

Correspondence Address: GREENLEE WINNER AND SULLIVAN P C, 4875 PEARL EAST CIRCLE, SUITE 200, BOULDER, CO, 80301, US

(21) Appl. No.: 10/494,163

(22) PCT Filed: Nov. 15, 2002
(86) PCT No.: PCT/US02/36712

Related U.S. Application Data
(60) Provisional application No. 60/339,842, filed on Nov. 16, 2001.

(51) Int. Cl. C12Q 1/48; C12N 9/00

(52) U.S. Cl. 435/15; 435/183

(57) ABSTRACT

The present invention provides a simple and efficient method of preparing an analyte-enzyme conjugate where the enzyme contains free, surface-accessible carboxyl moieties without generating undesired, cross-linked enzymes, while preserving CN the functionality of the enzyme. The method involves treating an enzyme with a blocking agent such that the free carboxyl moieties become non-reactive prior to the conjugation reaction with the desired analyte. The yield of the analyte-enzyme conjugate and the purity of the conjugates formed are high since cross-linking of the blocked enzymes is minimized or prevented. The invention is generally useful in preparing any conjugates of an analyte of interest and an enzyme containing surface accessible carboxyl moieties. The invention is particularly useful in preparing conjugates of any analyte of interest and horseradish peroxidase, alkaline phos-phatase or acetylcholine esterase. The conjugates prepared according to the invention are useful in a variety of assays including but not limited to enzyme-linked immunosorbent assay (ELISA), immunohistochemistry, and the like. The invention is further directed to analyte-enzyme conjugates prepared by the inventive method and to kits which contain an analyte-enzyme conjugate prepared by the methods herein for the detection and/or quantitation of an analyte in a sample.

### CROSS REFERENCE TO RELATED APPLICATIONS

This application claims priority from U.S. Provisional Patent Application No. 60/339,842, filed Nov. 16, 2001, which is incorporated herein in its entirety.

### FIELD OF THE INVENTION

The present invention relates to analytical reagents and methods of making such reagents, and particularly to improved processes of selectively preparing a conjugate of an analyte-enzyme without generating undesired multimers of the enzyme via cross-linking. The invention also relates to conjugates prepared by the processes.

### BACKGROUND OF THE INVENTION

Although there have been continuous efforts to improve various aspects of the assays that are commonly used in clinical medicine and research laboratories, there still exists a significant problem of preparing a large quantity of an analyte-enzyme conjugate of high purity by a simple and efficient process. For example, in a typical enzyme-linked immunosorbent assay (ELISA), given analyte-enzyme conjugates are prepared by direct conjugation of the analytes and the enzymes. In such conjugation reactions, when carboxyl groups in the enzyme are free to be conjugated, a large amount of cross-linking between enzyme molecules occurs under the reaction conditions (e.g., in the presence of carbodiimide), and few non-cross linked enzyme molecules are available to be conjugated to the analyte to form discrete analyte-enzyme conjugates. Therefore, it is necessary to carry out laborious chromatographic purification steps to obtain the analyte-enzyme conjugates separated from cross-linked materials and as a result only a small amount of the conjugate can be purified at a time.

Therefore, there exists a need for a more efficient method for preparing various analyte-enzyme conjugates that are essential components of numerous assays currently in use including ELISA.

Methods for the conjugation of analytes to proteins are generally known in the art using such reagents as hetero or homobifunctional crosslinkers containing N-maleimide, N-succinimide or hydrizides moieties which are somewhat selectively reactive with sulfhydryl groups, amino groups or aldehyde groups respectively. [Aslam and Dent (1998) Bioconjugation in Protein Coupling Techniques for the Biomedical Sciences, McMillan Ltd., London, UK.] Methods are also generally known in the art for blocking carboxyl groups of proteins [Hermanson, G. T. (1996) Bioconjugate Techniques, Academic Press, SanDiego, Calif., p. 135; Hoare et al. (1967) J. Biol. Chem. 242:2447-2453; Wen et al. (1999) J. Protein Chem., 18(6):677-86; Levesque, G. et al. (2000) Biomacromolecules, 1(3):387-399.] Those of ordinary skill in the art would not expect, in general, that art known blocking methods could be employed in forming analyte-enzyme conjugates without loss of significant enzyme activity which would render the analyte-conjugates undesirable or not useful in assays. The methods herein provide a general solution to the problem of making analyte-enzyme conjugates that retain substantial activity and high yield.

### SUMMARY OF THE INVENTION

The present invention is a simple and efficient method of conjugating an analyte to an enzyme that substantially eliminates the cross-linking of the enzyme by employing a pretreatment step of blocking surface accessible carboxyl moieties in the enzyme peptide prior to the conjugation reaction.

The method involves treating an enzyme containing free, surface-accessible carboxyl moieties with a blocking agent such that the free carboxyl moieties become non-reactive prior to the conjugation reaction with the desired analyte. Since cross-linking of the blocked enzymes is minimized or prevented, the yield of the analyte-enzyme conjugate formation and the purity of the conjugates formed are significantly improved compared to prior art methods.

As exemplified herein, the enzymes modified (i.e., blocked) according to the invention preferably exhibit no significant decrease in catalytic properties. A significant decrease in catalytic properties of an enzyme on modification is one in which one or more measurable indicators of catalytic activity of the modified (blocked) enzyme are decreased more than about 50% compared to the non-modified (non-blocked) enzyme by the modification employed. Therefore, the invention can be applied to improve the preparation of any analyte-enzyme conjugate of any analyte where the enzyme contains surface accessible carboxyl moieties that may trigger self aggregation of the enzyme molecules and thus reduce efficiency of analyte-enzyme conjugate formation.

Blocking agents useful in practicing this invention include, but are not limited to ammonia, molecules containing one or more primary amines, secondary amines or salts thereof. Amines that can be used as blocking agents include among others, ammonia or ammonium salts, taurine (or salts thereof), and tris(hydroxylmethyl)aminomethane (or salts thereof). The blocking agent is coupled to the free, surface-accessible carboxyl groups of the enzyme employing a coupling reagent that functions to covalently couple an amine to a carboxyl groups. Coupling reagents useful in this invention include, among others, carbodiimides, Woodward's Reagent K (N-ethyl-3-phenylisoxazolium-3′-sufonate) and CDIs (N,N′-carbonyldiimidazoles).

Preferred coupling reagents and blocking groups are at least partially water-soluble for improved efficiency of reaction. Further, the coupling reagent, such as a carbodiimide, can be bound to a solid support, such as a polydextran or agarose polymers. Carbodiimides, particularly those that are at least partially water-solub le are preferred coupling reagents. Carbodiimides useful in the methods of this invention, include, but are not limited to, 1-[3-dimethylaminopropyl]-3-ethylcarbodiimide methiodide (and other forms of EDC that are water-soluble) and 1-cyclohexyl-3-(2-morpholinoethyl)carbodiimide-methyl-p-toluenesulfonate. In a specific embodiment, the use of a carbodiimide (or other coupling reagent) carrying at least one bulky group is preferred for use in blocking enzymes in which one or more carboxyl groups may be associated with the enzyme active site. In another specific embodiment, the use of a carbodiimide (or other coupling reagent) bound to a solid support or polymer group is preferred for use in blocking enzymes in which one or more carboxyl groups may be associated with the enzyme active site. Carbodiimides having at least one bulky group include those that contain a cyclic alkyl or cyclic heteroakyl group, such as a cyclohexyl group, or a morpholino group.

The analyte conjugates prepared according to the invention can be employed in a variety of biochemical techniques and assays including, but not limited to, enzyme-linked irnmunosorbent assays (ELISA), competitive immunoassays, in situ chemical staining and immunohistochemical assays.

The invention is directed to analyte-enzyme conjugate prepared according to the processes disclosed herein and particularly to those in which all free, surface-accessible carboxyl groups are blocked in the enzyme. The invention is more specifically directed to analyte-enzyme conjugates where the enzyme is a peroxidase, a phosphatase, an esterase or a galatosidase. The invention is further specifically directed to analyte-enzyme conjugates where the enzyme is horseradish peroxidase, an alkaline phosphatase or an acetylcholine esterase. The invention includes analyte-enzyme conjugates made by the methods herein which are bound to a solid support, such as a polymer,

The invention is further directed to analyte-enzyme conjugates wherein the number of analyte molecules conjugated to each enzyme molecule is controlled so that the amount of analyte present can be determined by assaying for enzyme activity. More specifically the invention is directed to analyte-enzyme conjugates having one analyte molecule conjugated to each enzyme. More specifically the invention is directed to analyte-enzyme conjugates having more than one analyte molecule conjugated to each enzyme.

The invention also provides an assay kit for detection of an analyte which comprises one or more analyte-enzyme conjugates of this invention. Assay kits of this invention can further comprise one or more reagents for assaying the activity of the enzyme of the analyte enzyme conjugate in the kit. Assay kits of this invention can further comprise buffers, positive and/or negative controls and instructions for carrying out the assay.

### BRIEF DESCRIPTION OF TIHE INVENTION

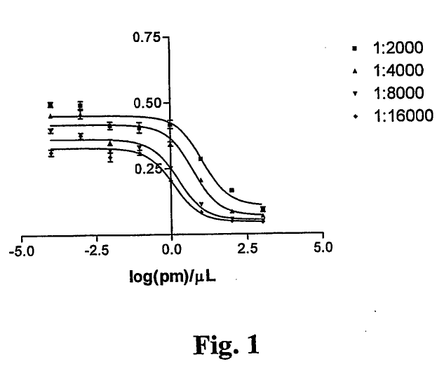

FIG. 1 shows the results of a cAMP assay using conjugates prepared according to the method disclosed herein. The X axis is the logarithmic concentration of cAMP and the Y axis is absorbance at 405 nm.

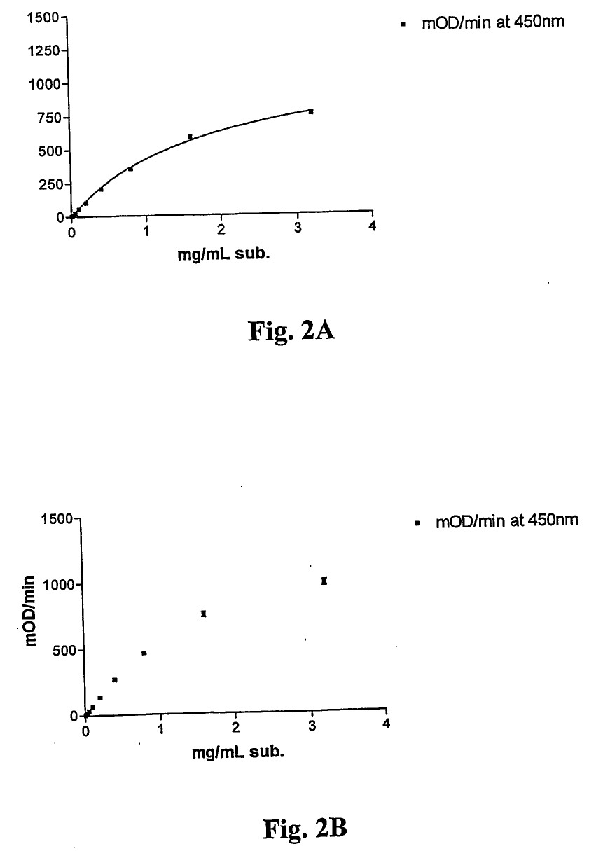

FIGS. 2A and 2B show a kinetic comparison of equal concentrations of unmodified (FIG. 2A) and modified (FIG. 2B) horseradish peroxidase (P) enzyme. The X axis is the substrate concentration and the Y axis is the absorbance read as mOD/min at 450 nm.

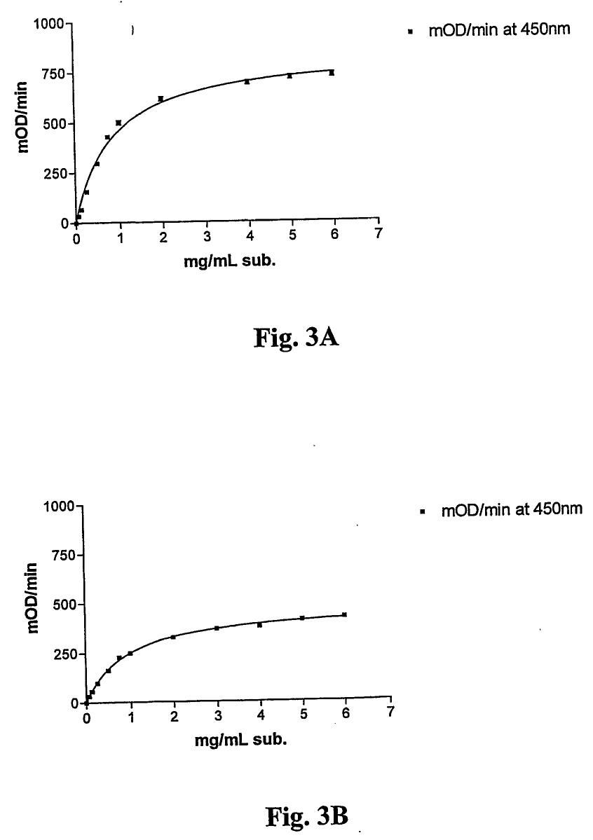

FIGS. 3A and 3B show a kinetic comparison of equal concentrations of unmodified (FIG. 3A) and modified (FIG. 3B) alkaline phosphatase (AP) enzyme. The X axis is the substrate concentration and the Y axis is the absorbance read as mOD/min at 450 mn.

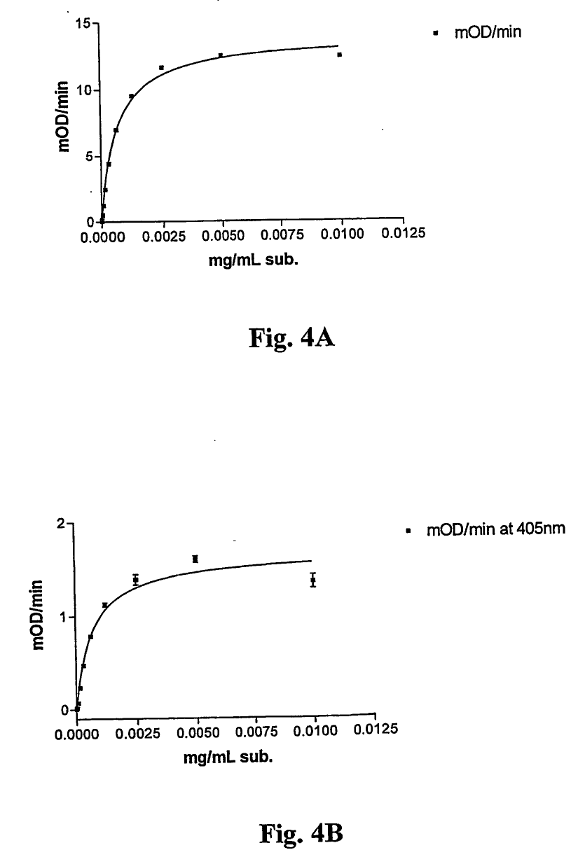

FIGS. 4A and 4B show a kinetic comparison of equal concentrations of unmodified (FIG. 4A) and modified (FIG. 4B) acetylcholine esterase (AChE). The X axis is the substrate concentration and the Y axis is the absorbance read as mOD/min at 405 nm.

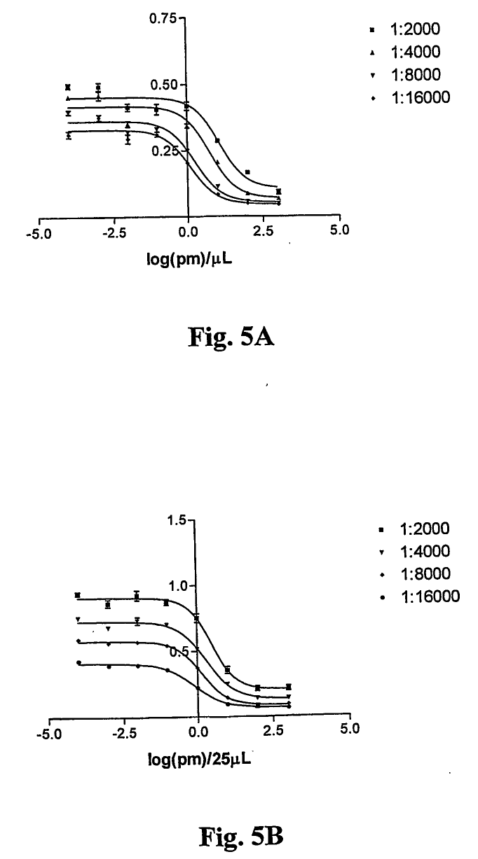

FIGS. 5A and SB show examples of cANP (FIG. SA) and cGMP (FIG. 5B) assays using cAMP-and cGMP-HRP conjugates prepared according to the invention.

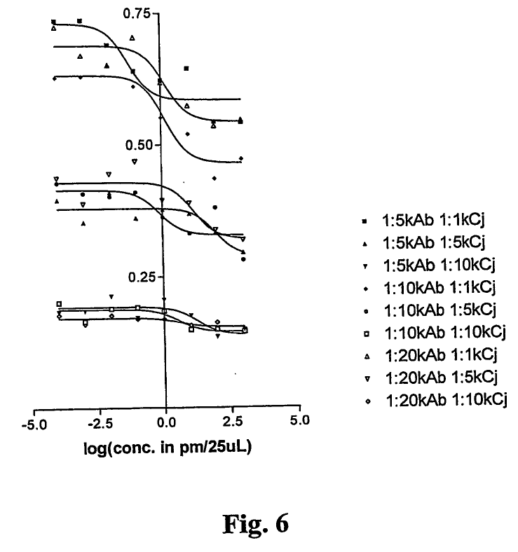

FIG. 6 shows the results of a Dippu-DH31 assay using a Dippu-DH31-HRP conjugate prepared according to the invention.

### DETAILED DESCRIPTION OF THE INVENTION

Unless otherwise stated herein, the terms and phrases used herein have their art-recognized meaning which can be found by reference to standard texts, journal references and contexts known to those skilled in the art. The following definitions are provided to clarify their specific use in the context of the invention.

The term, “analyte”, as used herein, refers to a molecule whose presence, absence or the level thereof in a given sample is to be determined. An analyte of interest thus means the analyte that is being assayed in a sample. Multiple analytes may be assessed in a single assay. An analyte that can be conjugated with an enzyme according to the invention includes, but is not limited to, cyclic nucleotides such as cAMP and cGMP, peptides, hormones, antibodies, any metabolites present in biological fluids and the like. The term enzyme refers generally to a catalytic peptide (or protein) and more specifically to a catalytic peptide (or protein) that is capable of generating an easily measurable signal (e.g., chromogenic or fluorogenic product) that is indicative of the concentration of the enzyme and analyte in the sample.

The term, “analyte-enzyme conjugate”, as used herein, refers to a molecule that has an analyte attached to a blocked enzyme prepared according to the method disclosed herein via a conjugation reaction. The conjugation is typically made through the formation of a covalent bond. Typically, the enzyme suitable for the invention is a “reporter” enzyme capable of generating a product that is easily measurable, i.e., calorimetric product, which because of the conjugation is indicative of the concentration of the analyte in a sample. Examples of enzymes useful for formation of conjugates, include, but are not limited to, peroxidases, particularly horseradish peroxidase (HRP), phosphatases, particularly alkaline phosphatase (AP), esterases, particularly acetylcholine esterase (AChE), and galactosidases, particularly beta-galactosidase.

A “blocking agent” is a molecule or portion thereof that is capable of rendering (i.e., blocking) a reactive moiety, particularly a reactive moiety on an enzyme, non-reactive through coupling to the reactive moiety. Accordingly, the type of reactive moiety to be rendered non-reactive determines the type of the blocking agent employed. For example, surface-accessible carboxyl moieties of an enzyme can be blocked by coupling to a blocking agent that renders the carboxyl moieties non-reactive for cross-linking (i.e. so that little or no enzyme-enzyme cross-links formed) in the subsequent conjugation reaction with an analyte. Examples of the blocking agents useful for the invention are ammonia, primary and secondary amines and salts thereof.

The term, “free, surface-accessible carboxyl moieties”, as used herein, refers to any carboxyl groups accessible for reaction, e.g. accessible for undesired cross-linkage on the surface (i.e., exposed) of the molecule, as opposed to being inaccessible, hidden, or not available for reaction. Free, surface-accessible carboxyl moieties can be modified to be non-reactive by the blocking agent in the pretreatment step described herein. Free, surface-accessible carboxyl moieties can be anywhere on the surface of the enzyme molecule, including the active site, as long as the modification does not significantly affect the enzyme activity. Based on the examples herein, the invention can be practiced with any enzyme having free, surface-accessible carboxyl group(s) without substantially affecting its catalytic activity. The invention is particularly useful for generating analyte conjugates of peroxidases, phosphatases, esterases and galactodidases, and more particularly is useful for generating analyte conjugates of HRP, AP and ACHE.

A “coupling reagent” is most generally a reagent that couples two reactive groups. As used herein the term is particularly directed to coupling reagents that couple an amine to a carboxyl moiety. The coupling reagents of this invention are used in particular to couple a blocking agent or portion thereof to a free carboxyl group to block that carboxyl group from reacting. More specifically, the preferred coupling reagents are carbodiimides, particularly those that are at least partially water-soluble. Coupling reagents other than carbodiimides include among others, Woodward's Reagent K (N-ethyl-3-phenylisoxazolium-3′-sufonate) and CDIs (N,N′-carbonyldiimidazoles).

Carbodiimides have the chemical formula R1-N═C═N—NR2, where R1 and R2 can be the same or different. Preferred R1 and R2 include alkyl groups which may be substituted with one or more hydrophilic (e.g., OH groups, amine or ammonium ion groups) or other substituent groups that do not detrimentally affect reactivity of the carbodiimide as a coupling reagent. R1 and R2 groups may be straight-chain, branched or cyclic and one or more CH or CH₂ groups may be replaced with a NR group, ⁺NRR′ group (where R and R′ are H or alkyl moieties), or an O or S atom. R1 or R2 can be cycloalkyl groups, such as cyclohexyl groups. R1 or R2 can be heterocyclic groups, such as morpholino groups. One or both of the R1 and R2 groups of the carbodiimide can be positively charged or be in the form of a salt. In general, however, any carbodiimide coupling reagent that is known in the art and is at least partially water-soluble can be used in the methods of the present invention.

The invention provides a method of preparing an analyte-enzyme conjugate where the enzyme contains free, surface-accessible carboxyl moieties. As a first step, an enzyme is treated with a blocking agent such as ammonia, a primary amine or a secondary amine or salts thereof such that after treatment the carboxyl moieties are not reactive in the subsequent conjugation reaction. The blocking agent is typically coupled to the carboxyl moiety employing a coupling reagent. The blocked enzyme is separated (if necessary or desired) and then subjected to the conjugation reaction with a desired analyte to produce an analyte-enzyme conjugate. In a preferred method the blocked enzyme is separated and purified employing one or more steps of dialysis. The pretreatment of the enzyme with the blocking agent minimizes or prevents the formation of undesirable cross-links between the enzyme molecules. The analyte-enzyme conjugate prepared by these methods has its free, surface-accessible carboxyl groups substantially blocked (in some cases in which one or more carboxyl moieties are associated with enzyme activity, these carboxyl moieties activity preferably remain unblocked).

The enzymes specifically exemplified herein include horseradish peroxidase, alkaline phosphatase, and acetylcholine esterase. However, the invention is applicable for any enzyme that contains free, surface-accessible carboxyl group(s).

Enzyme modification (i.e., blocking) as in the pretreatment step described herein has been considered by those in the art to be deleterious to the catalytic activity of the enzyme; however, modification to block free, surface-accessible carboxyl groups as described herein can yield an enzyme which retains measurable catalytic activity. Preferably, the modifications employed herein to block free, surface-accessible carboxyl groups do not substantially detrimentally change the catalytic properties of the enzyme, particular as measured by determination of the Km of the enzyme. A substantial detrimental change herein refers to modification of an enzyme such that it retains less than about 10% of the catalytic activity of the unmodified enzyme. In certain embodiments, the modifications of this invention do not result in an increase in Km of the modified enzyme greater than about 50% compared to the Km of the unmodified enzyme. In certain other embodiments, the modifications of this invention do not result in an increase in Km of the modified enzyme greater than about 20% compared to the Km of the unmodified enzyme. In yet other embodiments, the modifications of this invention do not result in an increase in Km of the modified enzyme greater than about 10% compared to the Km of the unmodified enzyme. In some cases, useful analyte-enzyme conjugates prepared by the methods herein may have Vmax that is significantly decreased by the modifications herein, yet which retain Km that is not substantially detrimentally changed by the modifications.

The modifications herein allow the facile and efficient synthesis of any analyte-enzyme conjugates that can be used in enzyme-linked assays, immunohistology assays or any other application that necessitates the conjugation of an enzyme to an analyte.

The present invention has broad adaptive potential in the development of biological assays as well as in organic synthesis. Any individual molecule which can be conjugated to a carrier molecule in order to raise antibodies and which can also be singly conjugated to an enzyme can be used in enzyme immuno assays. These assays have potential in clinical medicine as well as for research reagents.

Exemplified herein is a conjugate of an analyte to a specific functionality, such as an amino group, on an enzyme without cross-linking of enzyme molecules together, while maintaining enzymatic activity. The enzyme peptide was first blocked with a blocking agent such as ammonia or a primary or secondary amine (or a salt thereof), using an appropriate coupling method such as a solvent soluble (typically at least partially water-soluble) carbodiimide and a large excess of the blocking agent. The excess coupling reagent and blocking reagent were then removed, e.g., by dialysis, leaving a functional enzyme devoid of surface-accessible carboxyl groups. The analyte was then conjugated to the enzyme through a suitable functional group such as the terminal alpha-amino group, if conjugation of the enzyme to only a single analyte is desired, and/or to the amino group of an amino acid side chain, such as the side chain of lysine or arginine or a related basic amino acid, if conjugation of the enzyme to more that one analyte is desired.

The blocking agent can be selected to affect the surface properties, including for example, the solubility, of the enzyme. For example, the use of blocking agents with hydrophilic groups can be employed to increase the hydrophilicity of the enzyme surface. Alternatively, blocking groups with hydrophobic groups can be employed to increase the hydrophobicity of the enzyme surface.

Specifically exemplified herein are methods of modifying HRP by blocking surface-accessible free carboxyl groups, preparing conjugates of cAMP, cGMP, succinyl-cAMP, and [Cys₃₂]Dippu-DH31-Cys with the modified HRP, and the assay results using these conjugates. Of particular interest are conjugates of a single modified HRP with one molecule of the listed analytes or with a known and/or controlled number of molecules of a selected analyte. This invention also provides enzyme-conjugates formed by the methods described herein in which free, surface-accessible carboxyl groups are blocked and in which a selected number of molecules of analyte are conjugated to each enzyme molecule, those in which two molecules of an analyte are conjugated to each enzyme molecule and those in which 3, 4, 5, 6, 7, 8, 9 or 10 molecules of an analyte are conjugated to each enzyme molecule. The number of analytes conjugated to a given enzyme can be controlled by selection of the site in the enzyme for conjugation, by controlling conjugation reaction conditions or by choice of enzyme but are limited to the number of surface accessible amino groups on each enzyme.

FIGS. 2A and 2B demonstrate that the unmodified and modified horseradish peroxidase (HRP) according to the invention exhibit comparable kinetic parameters; Vmax of unmodified HRP is 1565 mOD/min and Km is 1.849 mOD and Vmax of modified (blocked) HRP is 1212 mOD/min and Km is 1.841 mOD.

FIGS. 3A and 3B demonstrate that the unmodified and modified alkaline phosphatase (AP) according to the invention exhibit comparable kinetic parameters; Vmax of unmodified AP is 851.9 mOD/min and Km is 0.8464 mOD and Vmax of modified (blocked) AP is 481.8 mOD/min and Km is 0.9295 mOD.

FIGS. 4A and 4B demonstrate that the unmodified and modified acetylcholine esterase (ACHE) according to the invention exhibit comparable kinetic parameters; Vmax of unmodified ACHE is 13.93 mOD/min and Km is 0.0006303 mOD and Vmax of modified AChE is 1.667 mOD/min and Km is 0.0007037 mOD.

FIGS. 5A and 5B demonstrate that the cAMP- and cGMP-HRP conjugates prepared according to the invention yield similar results in the cAMP and cGMP assays as compared to the cAMP- and cGMP-HRP conjugate prepared according to the conventional method.

FIG. 6 shows the results of a Dippu-DH31 assay using Dippu-DH31-HRP conjugates prepared according to the invention, demonstrating that the assay is functional. This is the first and only example of the Dippu-DH31 assay where Dippu-DH31 is directly conjugated to an enzyme.

The following examples are provided for illustrative purposes only, and are not intended to limit the scope of the invention as claimed herein. Any variations in the exemplified methods which occur to the skilled artisan are intended to fall within the scope of the present invention. More specifically, coupling reagents and blocking agents, other than those specifically exemplified are known in the art and can be employed in the practice of this invention without resort to undue experimentation. Additionally, methods and reagents for conjugating an analyte of interest to an enzyme other than those specifically exemplified are known in the art and can be employed in the practice of this invention without resort to undue experimentation. Reaction conditions (e.g., temperature, and pH) of coupling reaction and conjugation reactions can be adjusted as is known in the art dependent upon the specific coupling and conjugation reagents employed, and the specific enzymes and analytes that are to be conjugated.

### THE EXAMPLES

### Example 1

### Enzyme Modification

In order to block free carboxyl moieties on the horseradish peroxidase (HRP) enzyme, 4.5 mg of HRP (Biozyme Laboratories, San Diego, Calif. was dissolved in 1 ml of a mixture of 0.1M (2-[N-morpholino]ethanesulfonic acid) (used as a buffer) and 0.1M Tris (hydroxymethyl) aminomethane (a large excess of amine blocking agent) in purified dionized water that had been titrated to pH 4.75. Thereafter 15 mg of 1-[3-dimethyl-amino)propyl]-3-ethylcarbodiimide methiodide was added and the mixture was reacted for 2 hours at room temperature while stirring. Upon completion of the reaction, the mixture was dialyzed against 2 liters of PBS pH 7.4 overnight at 4° C. The modified enzyme was then removed from dialysis and assayed for activity using TMB (3,3′,5,5′-tetramethylbenzidine) peroxidase substrate [Porstman and Kiessing (1992) J. Immunol. Methods 150:521]. The details of the coupling reaction can be found in Grabarek, Z. et al. (1990) Anal. Biochem. 185:244-248; Williams, A. et al. (1981) J. Amer. Chem. Soc. 103:7090-7095; Gilles, M.A. et al. (1990) Anal. Biochem. 184:244.

A similar protocol is used to block alkaline phosphatase and acetylcholine esterase with the exception that 1-cyclohexyl-3-(2-morpholinoethyl)carbodinimde-methyl-p-toluenesulfonate is used instead of 1-[3-dimethylamino)propyl]-3-ethylcarbodiimide methiodide. This bulkier carbodiimide coupling reagent is employed to minimize inactivation of the enzyme. It is believed that the larger, bulkier coupling reagent (e.g., because of the presence of the cycloalkyl group and/or the morpholino group) cannot enter the enzyme active side as readily to react with carboxyl groups that are involved in enzyme activity. Other carbodiimides having such bulky R or R2 groups can be employed in blocking enzymes in which carboxyl moieties may be involved in enzyme catalytic activity.

### Example 2

### cAMP Assay

The following is an example of the use of an enzyme-conjugate prepared according to the invention in a cAMP Assay. [Horton et al. (1992) J. Immunological Meth. 15:31-40; Steiner et al. (1969) Proc. Natl. Acad. Sci. 64:367-373].

Affinity purified goat-anti-rabbit Fc antibody (27.5 μl) was added to 10 ml of phosphate buffered saline (PBS) (PBS is 0.15M NaCl, 0.01M sodium phosphate in purified water at pH 7.4) and made homogeneous. This solution (90 μl) was added to each well of a Costar High Binding EIA microtiter plate. The plate was then incubated for 4 hours at room temperature. The contents of the plate were discarded and 350 μl of a 3% solution of normal goat serum in PBS was added to each well. The plate was incubated at room temperature for 1 hour and the contents of the plate were discarded. Each well was then washed with 150 μl of washing buffer (washing buffer is 0.05% Tween 20 detergent in PBS). Thereafter 75 μl of a 1:10000 solution of affinity purified rabbit-anti-cAMP antibody in EIA buffer was added to each well. (EIA buffer is 0.15M NaCl, 0.02M sodium phosphate and 1 mM disodium EDTA in purified water at pH 7.4). Next, 25 μl of the cAMP solution to be quantified (dissolved in EIA buffer) and 25 μl of a cAN-BRP conjugate solution were added to each well. The cAMP-BRP conjugate solution was 2 μl of a 5 mg/ml stock solution of conjugate in 10 ml of EIA buffer. The plate was then allowed to incubate at 4° C. overnight. The following morning the plate contents were discarded and the plate was washed with washing buffer. TMB Peroxidase substrate (100 μl) was added and allowed to react until a purple color appeared. (TMI Peroxidase substrate was 50% part A and 50% part B; Part A was 0.4 grams per liter of 3,3′,5,5′-tetramethylbenzidine in an organic base and Part B was 0.02% hydrogen peroxide in an aqueous citric acid buffer.) The reaction was then quenched with 100 μl of 1M phosphoric acid. The colored product was read using a spectrophotometric plate reader measuring absorbance of light at a wavelength of 405 nanometers.

This assay is a competitive assay in which the capture antibody (goat-anti-rabbit) binds to the bottom of the well in the microtiter plate, the primary antibody (rabbit-anti-cAMP) binds to the capture antibody and then equilibrium is established between binding of free cAMP and cAMP-enzyme conjugate to the antibody specific for cAMP. The more free cAMP that is present the less cAMP conjugate that can bind to the antibody and the reverse is also true. Essentially, there is a “competition” for binding sites of the antibody. The amount of free cAMP in the sample is inversely proportional to the amount of colored product produced by the enzyme. A standard curve is generated with known concentrations of cAMP and the amount in the sample (unknown) can be compared to the standard curve to determine the concentration of cAMP in the sample. The standard curve is typically a logarithmic curve with cAMP concentrations ranging from 1000 picomoles per 25 microliters to 0.001 picomoles per 25 microliters. (see FIG. 1).

### Example 5

### Preparation of Succinyl-cAMP-RP Conjugate

Succinyl-cAMP was conjugated to blocked horseradish peroxidase prepared as described above, using 1-[3-dimethylamino)propyl]-3-ethylcarbodiimide methiodide (EDC) and N-hydroxysulfosuccinimide (Sulfo-NHS).

Approx. 1.366 mg of 2-O-monosuccinyl-cAMP and approx. 3.5 mg of N-hydroxysulfosuccinimide were added to the above horseradish peroxidase solution (˜4.6 mg in 1.5 ml PBS, pH 7.4). Approximately 13 mg of 1-[3-dimethylamino)propyl]-3-ethylcarbodiimide methiodide was then added and the solution was mixed on an Orbital shaker overnight at 4° C. The modified enzyme was dialyzed against 1 liter of PBS overnight four times. Thimerosal (0.01%) was added to the enzyme conjugate and stored at 4° C.

2-O-monosuccinyl-cGMP can be coupled to the HRP in a similar manner.

### Example 6

### Preparation of rCys₃₂]Dippu-DH31-HRP Conjugate

An insect hormone, [Cys₃₂]Dippu-DH31 Furuya, K. et al. (2000) Proc. Natl. Acad. Sci., 97:6469-6474] was conjugated to the blocked HRP as follows:

Blocked horseradish peroxidase prepared as above was introduced into a screw-cap eppendorf tube. N-[gamma-maleimidobutrryloxy]sulfosuccinimide ester (“sulfo-GMBS”) (1.3 mg) was then added and the combination mixed for 1 hour at room temperature. Na₂EDTA (0.037 g) was then added to make the solution 0.1M in EDTA to protect from heavy metals. The resulting solution was then shaken for an additional 60 min at room temperature.

A size exclusion column was prepared to purify unreacted sulfo-GMBS from HRP. Ten grams of Sephadex G-25 (Pharmacia, Piscataway, N.J.) was hydrated with 75 ml of deionized and purified water for 1 hour while shaking at 45° C. The slurry was aspirated to draw off fines and 50 ml of deionized and purified water was added again and the process was repeated until all fines were removed. An additional 50 ml of deionized and purified water was then added and the mixture was evacuated for 30 min. The gel was again aspirated and the slurry was poured into a glass chromatography column 1.5 cm×10 cm. The modified enzyme was loaded and eluted with PBS, pH 7.4. Dippu 31-Cys (1 mg) was then added to the eluted fraction and reacted overnight at 4° C. on a rotary rocker.

All references cited in the present application are incorporated by reference in their entirety herein.

1. A process of preparing an analyte-enzyme conjugate wherein said enzyme contains one or more free, surface-accessible carboxyl moieties, comprising the steps of:
a. treating the enzyme with an amine and a coupling agent under conditions suitable for coupling a carboxyl moiety to an amine to generate a blocked enzyme; and
b. conjugating one or more analyte molecules to the blocked enzyme to form the analyte-enzyme conjugate wherein the blocked enzyme retains measurable catalytic activity.

2. The process of claim 1 wherein the blocked enzyme exhibits no substantial decrease in catalytic activity compared to the enzyme.

3. The process of claim 2 wherein the analyte-enzyme conjugate is produced with little or no cross-linked multimeric enzyme molecules.

4. The process of claim 3 further comprising a step of separating the blocked enzyme from the reaction mixture prior to conjugation.

5. The process of claim 1 wherein the amine is selected from the group consisting of ammonia, a primary amine, a secondary amine, and salts thereof.

6. The process of claim 5 wherein the coupling agent is a carbodiimide.

7. The process of claim 5 wherein the coupling agent is water-soluble.

8. The process of claim 6 wherein the coupling agent is water-soluble.

9. The process of claim 8 wherein the coupling agent is bound to a solid support.

10. The process of claim 9 wherein the coupling agent is 1-cyclohexyl-3-(2-morpholinoethyl)carbodiimide-methyl-p-toluenesulfonate.

11. The process of claim 9 wherein the coupling agent is 1-[3-dimethylaminopropyl]-3-ethylcarbodiimide methiodide.

12. The process of claim 11 wherein the analyte is conjugated to one or more amine groups on the blocked enzyme

13. The process of claim 12 wherein the amine group is the N-terminal amine group of the enzyme.

14. The process of claim 12 wherein the amine group is one or more amine groups of an amino acid side chain of the enzyme.

15. The process of claim 14 wherein the amine group is an amine group of lysine or arginine.

16. The process of claim 12 wherein the analyte is conjugated to the enzyme using a N-hydroxysuccinimide.

17. The process of claim 12 wherein the analyte is conjugated to the enzyme using a N-hydroxysulfosuccinimide.

18. The process of claim 12 wherein the analyte is conjugated to the enzyme using N-[gamma-maleimidobutyryloxy]sulfosuccinimide.

19. The process of claim 1 wherein the enzyme is coupled to more than one amine.

20. The process of claim 1 wherein said enzyme is selected from the group consisting of a peroxidase, a phosphatase and an esterase.

21. The process of claim 20 wherein the enzyme is selected from the group consisting of horseradish peroxidase, alkaline phosphatase and acetylcholine esterase.

22. The process of claim 21 wherein said enzyme is horseradish peroxidase.

23. The process of claim 21 wherein said enzyme is alkaline phosphatase.

24. The process of claim 21 wherein said enzyme is acetylcholine esterase.

25. The process of claim 4 further comprising a step of separating the blocked enzyme from the reaction mixture by dialysis prior to conjugation.

26. The process of claim 1 wherein the analyte is selected from the group consisting of cyclic adenosine monophosphate (cAMP), cyclic guanosine monophosphate (cGMP), a peptide, a hormone, an antibody and [Cys₃₂]Dippu-DH31.

27-31. (canceled).

32. The process of claim 5 wherein the amine is tris(hydroxymethyl)aminomethane.

33. The process of claim 5 wherein the amine is ammonia.

34. The process of claim 1 wherein the amine carries one or more hydrophilic groups.

35. The process of claim 1 wherein the amine carries one or more hydrophobic group.

36. The process of claim 5 wherein the amine that is conjugated to the blocked enzyme is bound to a solid support.

37. An analyte-enzyme conjugate prepared according to the process of claim 1.

38. An analyte-enzyme conjugate wherein all free, surface-accessible carboxyl groups are blocked in the enzyme.

39. An analyte-enzyme conjugate of claim 38 wherein the enzyme is selected from the group consisting of horseradish peroxidase, alkaline phosphatase and acetylcholine esterase.

40. An analyte-enzyme conjugate of claim 39 which is bound to a solid support.

41. An analyte-enzyme conjugate of claim 40 wherein a single analyte is conjugated to each enzyme.

42. An analyte-enzyme conjugate of claim 40 wherein more than one analyte is conjugated to each enzyme.

43. An analyte-enzyme conjugate of claim 40 wherein the number of analytes conjugated to each enzyme is controlled.

44. An analyte-enzyme conjugate of claim 40 wherein the blocked enzyme is conjugated to the analyte using a N-hydroxysuccinimide.

45. An assay kit for detection of an analyte which comprises one or more analyte-enzyme conjugates of claim 38.

46. An assay kit of claim 45 further comprising a reagent for assaying the activity of the enzyme of the analyte-enzyme conjugate.
